(19) United States
(12) Patent Application Publication
(10) Pub. No.: US 2005/0288540 A1
(43) Pub. Date: Dec. 29, 2005

(54) Process for the preparation of a hydrocarbon product stream being rich in C6 and C7 iso-paraffins

(76) Inventor: Sven Ivar Hommeltoft, Hillerod (DK)

Correspondence Address: DICKSTEIN SHAPIRO MORIN & OSHINSKY LLP, 2101 L Street, NW, Washington, DC, 20037, US

(21) Appl. No.: 11/167,592

(22) Filed: Jun. 28, 2005

(30) Foreign Application Priority Data
Jun. 28, 2004 (DK) PA 2004 01014

(51) Int. Cl. C07C 6/08

(52) U.S. Cl. 585/708

(57) ABSTRACT

Process for the preparation of a hydrocarbon product stream being rich in C6 and C7 paraffinic hydrocarbons by reaction of a feed stream comprising iso-C5 and C8⁺ paraffins in presence of a strong acid catalyst.

The present invention relates to a process for the preparation of a hydrocarbon product stream being rich in C6 and C7 isoparaffins by catalytic treatment of a feed stream comprising iso-C5 and C8⁺ paraffins in presence of a strong acid catalyst.

Isopentane is because of its vapour pressure not always desirable as blending component in gasoline and it is therefore desirable to find ways to convert it into heavier isoalkanes, which have lower vapour pressure. One way to convert isopentane to heavier material is to alkylate it with light olefins to form alkylate. However, isopentane alkylate typically has poor quality. It has poor octane value and contains heavy boiling material, which may be difficult to blend into gasoline because of specifications limiting the content of heavier material in gasoline. In general, there are requirements to the end point of the gasoline and in some areas (California) there are specifications defining a maximum temperature at which 50% of the gasoline distils (T₅₀). This later specification in practice limits the content of C8+hydrocarbons in the gasoline. In areas where specifications on T₅₀ apply, the refineries typically have a shortage of C6-C7.

We have found that it is possible in an acid catalysed process to react octane and heavier paraffins and isoparaffins with isopentane to form a mixture of isoparaffins boiling between isopentane and the heavy paraffin and isobutane. Since isobutane reacts very similar as isopentane, it may be recycled to react with more of the heavy paraffins or it may be withdrawn from the process and used elsewhere for instance for isobutane alkylation.

Suitable catalysts include liquid super acids such as mixtures of trifluoromethanesulphonic acid or fluorosulfonic acid with strong Lewis acids such as antimony pentafluoride. These catalysts typically operate in the range 0-50° C. at atmospheric pressure or at low to moderate pressure. Hydrogen is not required. Trifluoromethanesulfonic acid and fluorosulfonic acid show catalytic activity in the absence of strong Lewis acid though the activity is less.

In a preferred embodiment of the process a feed consisting of 20-80% of a mixture of 0-100% isobutane in iso-pentane and 20-80% of a mixture comprising C8-C10 paraffinic hydrocarbons is fed to the reactor, where it is reacted under suitable reaction conditions to produce a mixture of hydrocarbons enriched in C6-C7 iso-paraffins. The effluent from the reactor is preferably separated and the C6-C7 hydrocarbons withdrawn as product while C8 and heavier hydrocarbons as well as isopentane and isobutane is recycled to the reactor. Optionally isobutane may be withdrawn from the process as a second product.

Optionally the feed to the process may be withdrawn from another process that converts naphthenic hydrocarbons and remove aromatic hydrocarbons in such a way the content of naphthenic and aromatic hydrocarbons are minimised.

### EXAMPLES

### Example 1

Reaction of n-Octane with isopentane Catalysed by a Mixture of Fluorosulfonic Acid and Antimony Pentafluoride (Magic Acid)

10 ml (31 g, 143 mmoles) SbF₅ and 10 ml (17 g, 174 mmoles) FSO₃H is mixed in a 100 ml flask under nitrogen and cooled to −30° C. 10 ml n-octane and 10 ml isopentane were added and the temperature increased to 0° C. After 1 hr additional 20 ml isopentane and 20 ml n-octane were added. 15 min after the last addition, a sample of the hydrocarbon phase was withdrawn and isolated by GC. The sample contained 9.1 wt % C4, 19.0% C5, 11.8% C6-C7, 59.1 wt % C8, 0.7 wt % C9 and 0.3 wt % C10+.

### Example 2

Reaction of n-Nonane and Isopentane Catalysed by a Mixture of Fluorosulfonic acid and Antimony Pentafluoride (Magic Acid)

The catalyst used in Example 1 was reused after decanting off the hydrocarbon phase and washing the acid phase with isopentane. This catalyst was cooled to −30° C., a mixture of 30 ml n-nonane and 30 ml isopentane were added and the temperature rose to 0° C. A sample of the hydrocarbon phase was taken after 1 hr. The sample contained 12.3 wt % C4, 13.4% C5, 16.7 wt % C6-C7 (nearly half of the isohexanes was the high-octane 2,2-dimethylbutane), 2.7 wt % C8, 53.2 wt % C9 and 1.7 wt % C10+.

1. Process for the preparation of a hydrocarbon product stream being rich in C6 and C7 paraffinic hydrocarbons by reaction of a feed stream comprising iso-C5 and C8+paraffins in presence of a strong acid catalyst.

2. Process according to claim 1, wherein the catalyst is selected from group consisting of a combination of a fluorosulfonic acid with a strong Lewis acid.

3. Process according to claim 1, wherein the strong acid catalyst is trifluoromethanesulfonic acid and/or fluorosulfonic acid combined with antimony pentafluoride.

4. Process according to claim 1, wherein the C6 and C7 paraffinic hydrocarbons are separated from the product stream and reminder of the product stream is combined with the feed stream.

5. Process according to claim 1, wherein iso-C4 further contained in the product stream is withdrawn from the product stream.

6. Process according to claim 1, wherein the iso-C5 comprising process stream further comprises iso-C4.
